(19) United States
(12) Patent Application Publication
(10) Pub. No.: US 2025/0000420 A1
(43) Pub. Date: Jan. 2, 2025

(54) INTEGRATED PULL RING AND COUPLER FOR MEDICAL DEVICES

(71) Applicant: St. Jude Medical, Cardiology Division, Inc., St. Paul, MN (US)

(72) Inventors: Troy Tegg, Elk River, MN (US); Wayne Neudecker, Brooklyn Park, MN (US)

(21) Appl. No.: 18/673,572

(22) Filed: May 24, 2024

Related U.S. Application Data
(60) Provisional application No. 63/511,048, filed on Jun. 29, 2023.

(51) Int. Cl. A61B 5/287; A61B 5/00

(52) U.S. Cl. CPC A61B 5/287; A61B 5/6852; A61B 2560/0468; A61B 2562/0223

(57) ABSTRACT

A catheter (e.g., a deflectable electrophysiology catheter) includes a shaft, a tip electrode, and a coupler joining the tip electrode and the shaft. The coupler includes a cylindrical body having an annular wall defining a central cavity, a plurality of vias extending through the annular wall into the central cavity, a pull wire receptacle, and a sensor receptacle. A pull wire is connected at its distal end to the coupler within the pull wire receptacle; a sensor (e.g., a localization sensor) is secured within the sensor receptacle. Ring electrodes may be positioned around the coupler; corresponding conductors may be routed therefrom, through the vias, and into the central cavity.

### CROSS-REFERENCE TO RELATED APPLICATIONS

This application claims the benefit of U.S. provisional application No. 63/511,048, filed 29 Jun. 2023, which is hereby incorporated by reference as though fully set forth herein.

### BACKGROUND

The instant disclosure relates to catheters for use in medical procedures, such as electrophysiology studies. In particular, the instant disclosure relates to an integrated pull ring and coupler that can be used both to join a catheter shaft to a catheter tip, such as a tip electrode, and to secure pull wires to enable deflection of the catheter tip, in an electrophysiology catheter.

Catheters are used for an ever-growing number of procedures, such as diagnostic, therapeutic, and ablative procedures, to name just a few examples. Typically, the catheter is manipulated through the patient's vasculature and to the intended site, for example, a site within the patient's heart.

A typical electrophysiology catheter includes an elongate shaft and one or more electrodes on the distal end of the shaft. The electrodes may be used for ablation, diagnosis, or the like. Oftentimes, these electrodes include a tip electrode at the distal end of the elongate shaft. The tip electrode may be secured to the shaft via a coupler.

The distal end of the catheter may also be deflectable—that is, capable of assuming various degrees of curvature in one or more planes. As those of ordinary skill in the art will appreciate, deflection of the distal end of the catheter can be achieved through the use of one or more pull wires. In particular, the proximal end of each pull wire is secured to a suitable actuator at the proximal end of the catheter, while the distal end of each pull wire is secured near the distal end of the catheter, often by attaching the pull wire to a pull ring that is embedded in the wall of the catheter shaft. Actuation of the actuator places one or more of the pull wires in tension; this, in turn, causes a corresponding deflection at the distal end of the catheter.

It is also known to incorporate various sensors near the distal end of electrophysiology catheters. As one example, one or more temperature sensors, such as thermocouples or thermistors, can be positioned to measure the temperature of tissue undergoing ablation. As another example, the distal end of the catheter can incorporate magnetic coils to enable an electroanatomical mapping system to determine the position and orientation of the catheter during an electrophysiology study.

Manufacturing catheters that incorporate the foregoing features, however, can be a complex process. For example, in order for an electroanatomical mapping system to be able to determine the position and orientation of the tip electrode accurately, the corresponding sensors (e.g., magnetic coils) must be placed with precision during catheter assembly.

### BRIEF SUMMARY

Disclosed herein is a catheter including: a catheter shaft; a tip electrode; a coupler joining the tip electrode to the catheter shaft, wherein the coupler includes a cylindrical body having an annular wall that defines a central cavity, a plurality of vias extending through the annular wall into the central cavity, a pull wire receptacle, and a sensor receptacle; a pull wire having a distal end connected to the coupler within the pull wire receptacle; and a sensor secured within the sensor receptacle.

In embodiments of the disclosure, the sensor includes a localization sensor, such as a plurality of magnetic coils.

The sensor receptacle can include a slot that extends both along a longitudinal axis of the cylindrical body and around a circumference of the cylindrical body.

The pull wire receptacle can include a slot beginning at a proximal end of the cylindrical body and having a length extending along a longitudinal axis of the cylindrical body.

Optionally, the catheter can also include a ring electrode positioned around the coupler and a conductor attached to the ring electrode and routed through a via of the plurality of vias into the central cavity.

A handle can be secured to a proximal end of the catheter shaft, and the handle can include an actuator. A proximal end of the pull wire can be secured to the actuator such that movement of the actuator applies a tensile force to the pull wire and the tensile force is transmitted to the catheter shaft through the connection between the pull wire and the coupler.

Also disclosed herein is a method of manufacturing a catheter, including: providing a catheter shaft, a tip electrode, a pull wire, a sensor, and a coupler, wherein the coupler includes a cylindrical body having an annular wall that defines a central cavity, a plurality of vias extending through the annular wall into the central cavity, a pull wire receptacle, and a sensor receptacle; joining the catheter shaft, the tip electrode, and the coupler; connecting the pull wire to the coupler by securing a distal end of the pull wire in the pull wire receptacle; and securing the sensor within the sensor receptacle.

The sensor can include a localization sensor such as a plurality of magnetic coils.

According to aspects disclosed herein, the method also includes placing a ring electrode around the coupler; attaching a conductor to the ring electrode; and routing the conductor through a via of the plurality of vias and into the central cavity.

It is also contemplated that the method can include securing a handle to a proximal end of the catheter shaft, the handle including an actuator, and connecting the pull wire to the handle by securing a proximal end of the pull wire to the actuator such that movement of the actuator applies a tensile force to the pull wire and the tensile force is transmitted to the catheter shaft through the connection between the pull wire and the coupler.

The instant disclosure also provides a coupler for securing a tip electrode, a sensor, and a pull wire to a catheter shaft. The coupler includes: a cylindrical body having an annular wall that defines a central cavity; a plurality of vias extending through the annular wall into the central cavity; a pull wire receptacle; and a sensor receptacle.

The sensor receptacle can include a slot that extends both along a longitudinal axis of the cylindrical body and around a circumference of the cylindrical body.

The pull wire receptacle can include a slot that begins at a proximal end of the cylindrical body and has a length extending along a longitudinal axis of the cylindrical body.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts a representative electrophysiology catheter.

FIG. 2 is a view of the distal portion of the electrophysiology catheter of FIG. 1, with portions of the exterior removed in order to reveal aspects of the interior.

FIGS. 3A and 3B are perspective views of a coupler according to embodiments disclosed herein.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

For the sake of illustration, certain embodiments of the disclosure will be explained herein with reference to a deflectable electrophysiology (EP) catheter, such as a radiofrequency (RF) ablation catheter utilized in cardiac ablation procedures. One exemplary such catheter is the TactiFlex™ Ablation Catheter, Sensor Enabled™, of Abbott Laboratories (Abbott Park, IL). It should be understood, however, that the present teachings may be applied to good advantage in other contexts as well.

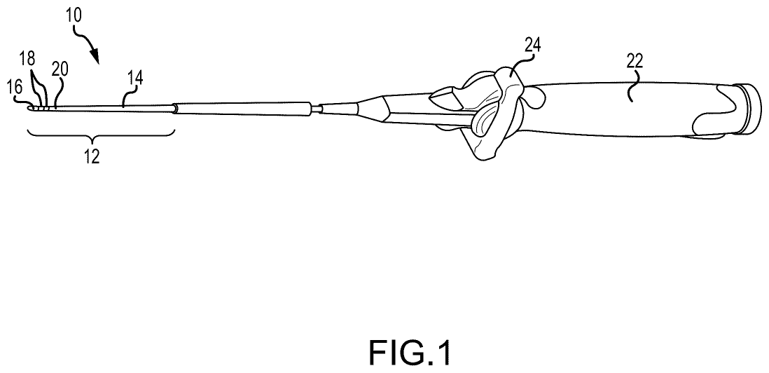

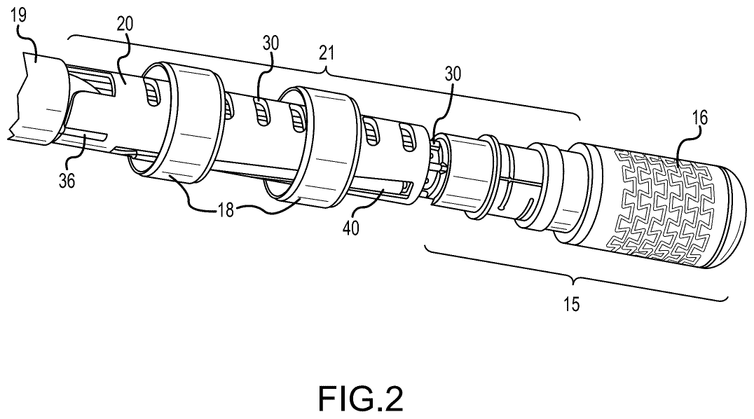

Referring now to the figures, FIG. 1 depicts an embodiment of an EP catheter 10 according to aspects of the present disclosure. Catheter 10 includes catheter body 12, which in turn includes a catheter shaft 14 and a tip assembly 15 (see FIG. 2), including a tip electrode 16, attached to the distal end of catheter shaft 14. One or more ring electrodes 18 may also be carried by catheter shaft 14 near its distal end.

In some embodiments, catheter shaft 14 is tubular (e.g., it defines at least one lumen therethrough). It should also be understood that the length of catheter shaft 14 as depicted in FIG. 1 is merely illustrative and may vary without departing from the scope of the instant disclosure. Of course, the overall length of catheter body 12 should be long enough to reach the intended destination within the patient's body (e.g., a location within the patient's heart).

Catheter shaft 14 will typically be made of a biocompatible polymeric material, such as polytetrafluoroethylene (PTFE) tubing (e.g., TEFLON® brand tubing). Of course, other polymeric materials, such as fluorinated ethylene-propylene copolymer (FEP), perfluoroalkoxyethylene (PFA), poly (vinylidene fluoride), poly (ethylene-co-tetrafluoroethylene), and other fluoropolymers, may be utilized. Additional suitable materials for catheter shaft 14 include, without limitation, polyamide-based thermoplastic elastomers (namely poly (ether-block-amide), such as PEBAX®), polyester-based thermoplastic elastomers (e.g., HYTREL®), thermoplastic polyurethanes (e.g., PELLETHANE®, ESTANE®), ionic thermoplastic elastomers, functionalized thermoplastic olefins, and any combinations thereof. In general, suitable materials for catheter shaft 14 may also be selected from various thermoplastics, including, without limitation, polyamides, polyurethanes, polyesters, functionalized polyolefins, polycarbonate, polysulfones, polyimides, polyketones, liquid crystal polymers and any combination thereof. It is also contemplated that the durometer of catheter shaft 14 may vary along its length.

Insofar as the basic construction of catheter shaft 14 will be familiar to those of ordinary skill in the art, it will not be discussed in further detail herein except to the extent necessary to understand the instant disclosure. For purposes of illustration only, however, U.S. Pat. No. 8,431,057, which is hereby incorporated by reference as though fully set forth herein, describes certain aspects of the construction of an illustrative catheter shaft 14, including an assembly that includes a tubular inner liner, a reinforcing layer, an outer layer, and the use of a reflow process to bond the various elements of the catheter assembly together under heat and pressure.

In FIG. 2, the outermost layer of catheter shaft 14 has been omitted in order to illustrate certain aspects of the disclosure that would otherwise be obscured. Thus, visible in FIG. 2 are the inner liner 19 of catheter shaft 14, a coupler 20, and tip assembly 15 including tip electrode 16. The term “pocket” is used herein to refer to the portion of catheter shaft 14, denoted by reference numeral 21, into which coupler 20 is inserted and secured, extending to the proximal end of tip electrode 16.

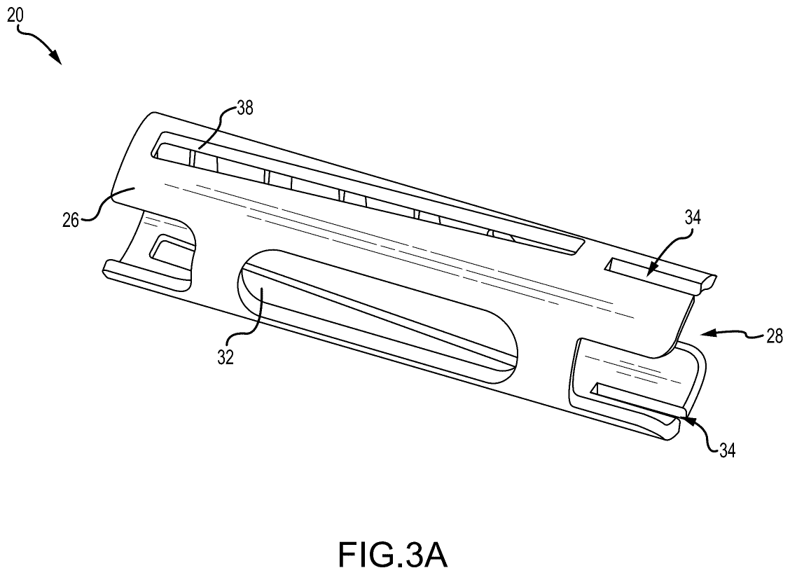

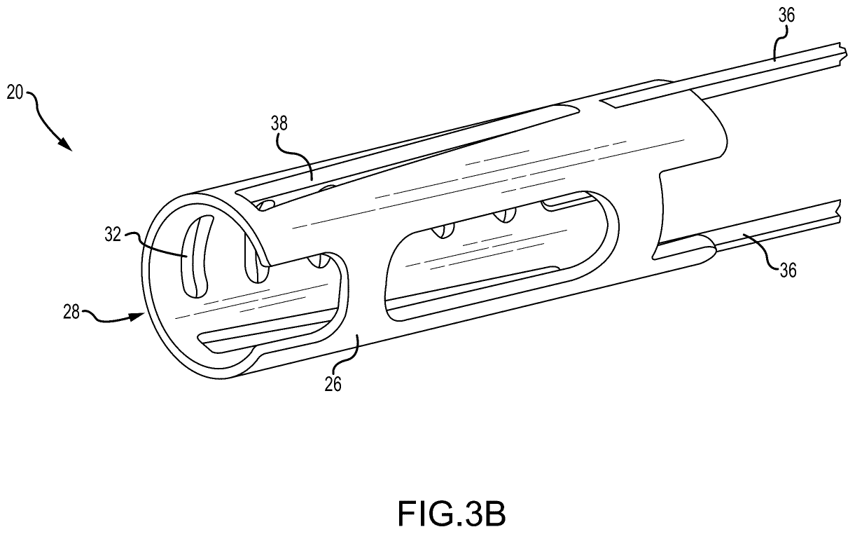

Suitable materials for coupler 20 include, without limitation, various grades of stainless steel, such as Type 304 or Type 316 stainless steel. Coupler 20 is described in further detail below with reference to FIGS. 3A and 3B.

A handle 22 is coupled to catheter body 12. Handle 22 can include a suitable actuator 24 to control the deflection of catheter body 12, for example as described in U.S. Pat. No. 8,369,923, which is hereby incorporated by reference as though fully set forth herein. Various handles and their associated actuators for use in connection with electrophysiology catheters are known, and thus handle 22 will not be described in further detail herein.

FIGS. 3A and 3B depict coupler 20. As shown therein, coupler 20 generally includes a cylindrical body including an annular wall 26 that defines a central cavity 28. For purposes of this disclosure, the phrase “cylindrical body” refers broadly to a body that is generally cylindrical, and is not limited to bodies that are perfectly cylindrical. Thus, a body can include grooves, ridges, flat portions, channels, discontinuities, and the like, as described in connection with certain embodiments disclosed herein, and yet still be a “cylindrical body” within the meaning of the present disclosure.

As visible in FIG. 2, which exposes the interior of catheter shaft 14, central cavity 28 can be used to pass other components of catheter 10, such as conductors 30 for tip electrode 16 and/or ring electrodes 18, through coupler 20 and into catheter shaft 14 (e.g., into the lumen(s) defined by catheter shaft 14). In some embodiments of the disclosure, these components enter and exit central cavity 28 through the openings at the proximal and distal ends of coupler 20.

It is also contemplated, however, that one or more components can pass into central cavity 28 through the vias 32 that extend through annular wall 26 into central cavity 28. For instance, while the conductor for tip electrode 16 may pass through the open distal end of coupler 20, it may be more convenient to string the conductors for ring electrodes 18 into central cavity 28 through vias 32. This simplifies the assembly of catheter 10 by eliminating the need to route conductors along the outer surface of annular wall 26.

Vias 32 also simplify the injection of epoxy (or other suitable potting compound) into central cavity 28 when adhesively securing coupler 20 within the pocket of catheter shaft 14. This limits the ability of fluid to enter the interior of catheter 10 through coupler 20.

It should be understood that the configuration (e.g., number, shape, spacing, layout, and the like) of vias 32 shown in FIGS. 2, 3A, and 3B is merely exemplary. Other configurations are regarded as within the scope of the present disclosure.

In addition to joining catheter shaft 14 to tip assembly 15, coupler 20 can replace stand-alone pull rings by including one or more pull wire receptacles 34 near its proximal end (that is, the end oriented towards handle 22). In some embodiments of the disclosure, pull wire receptacles 34 are formed as slots into annular wall 26 that begin at the proximal end of coupler 20 and have a length that extends along (that is, parallel to) the longitudinal axis of coupler 20. The distal ends of pull wires 36 can be connected to coupler 20 within respective pull wire receptacles 34, such as by soldering or another appropriate manufacturing technique.

As those of ordinary skill in the art will appreciate, pull wires 36 can then be routed proximally through inner liner 19 to the proximal end of catheter shaft 14, with the proximal ends of pull wires 36 secured to actuator 24 of handle 22 such that movement of actuator 24 places one or more pull wires 36 in tension (and may, in some embodiments, place other pull wires 36 in compression). These tensile (and, in some embodiments, compressive) forces are, in turn, transmitted to catheter shaft 14 through the connection between pull wire 36 and coupler 20, which causes deflection at the distal end of shaft 14 (though not within the portion of catheter 10 extending beyond the point at which pull wire 36 is secured to coupler 20, including pocket 21 and tip assembly 15, referred to herein as the “sweep,” which remains generally straight).

Coupler 20 also includes one or more sensor receptacles 38. Sensor receptacles 38 are positioned to place sensors 40 secured therein correctly (e.g., in the proper relationship to tip assembly 15). For instance, each sensor receptacle 38 can be formed as a slot that both extends along a (that is, parallel to) the longitudinal axis of coupler 20 and also wraps around a circumference of coupler 20.

In embodiments of the disclosure, sensors 40 are localization sensors. That is, they are sensors that can be used by an electroanatomical mapping system to measure the position and orientation of catheter 10. For example, the EnSite™ X Cardiac Mapping System of Abbott Laboratories is capable of generating a magnetic localization field; thus, sensors 40 can be magnetic coils that measure the components of this magnetic localization field to determine the position and orientation of catheter 10 therein. Of course, other localization modalities, including impedance-based localization modalities, are also contemplated.

One approach to assembling catheter shaft 14, tip assembly 15, and coupler 20 can also be understood with reference to FIGS. 2, 3A, and 3B. Although various steps will be described in a particular order herein, those of ordinary skill in the art will appreciate that the instant teachings are not limited to the order, or even to the particular approach, used herein for purposes of explanation. That is, the various steps described herein can be rearranged or substituted without departing from the scope of the present disclosure.

As mentioned above, the general construction of catheter shaft 14 will be familiar to those of ordinary skill in the art, and thus need not be described in further detail.

Pull wires 36, conductors 30, and other elements are routed through catheter shaft 14 (e.g., through inner liner 19) and/or coupler 20, in a stringing process that will also be generally familiar to those of ordinary skill in the art. The ordinarily-skilled artisan will appreciate, however, that coupler 20 according to the instant disclosure offers advantages during the stringing process. For example, the increased cross-sectional area of central cavity 28 makes it easier to route elements through coupler 20 relative to extant structures. Pull wires 36 and sensors 40 can then be secured to coupler 20 in their respective receptacles 34, 38, such as by welding or through the use of a suitable adhesive (e.g., epoxy).

Coupler 20 can then be inserted into pocket 21 of catheter shaft 14. Once again, coupler 20 according to the instant disclosure offers advantages over extant assemblies in this regard. For instance, by integrating a pull ring and a sensor coupler into a single component (i.e., coupler 20), the total number of components in catheter 10 is reduced, with a corresponding reduction in parts cost and production complexity.

Likewise, the length of coupler 20 increases the strength of catheter shaft 14 within pocket 21. Indeed, coupler 20 may reinforce pocket 21 to an extent that, if catheter shaft 14 includes a reinforcing layer (e.g., a braided wire mesh layer), it may terminate at or before pocket 21, such that the reinforcing layer does not extend over coupler 20. This has the further advantage of improving high-voltage applications of catheter 10 (e.g., pulsed field ablation) by minimizing the amount of electrically-conductive material at or near tip electrode 14.

Tip assembly 15 can then be inserted into pocket 21 (e.g., with the proximal end of tip assembly 15 abutting coupler 20 in some embodiments of the disclosure and inserted into central cavity 28 of coupler 20 in other embodiments of the disclosure). Catheter shaft 14, coupler 20, and tip assembly 15 can then be potted together with epoxy or another adhesive or potting compound.

Ring electrodes 18 can then be secured to catheter shaft 14 (e.g., by swaging).

Although several embodiments of this invention have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A catheter comprising:
a catheter shaft;
a tip electrode;
a coupler joining the tip electrode to the catheter shaft, wherein the coupler comprises:
a cylindrical body having an annular wall that defines a central cavity;
a plurality of vias extending through the annular wall into the central cavity;
a pull wire receptacle; and
a sensor receptacle;
a pull wire having a distal end connected to the coupler within the pull wire receptacle; and
a sensor secured within the sensor receptacle.

2. The catheter according to claim 1, wherein the sensor comprises a localization sensor.

3. The catheter according to claim 2, wherein the localization sensor comprises a plurality of magnetic coils.

4. The catheter according to claim 1, wherein the sensor receptacle comprises a slot that extends both along a longitudinal axis of the cylindrical body and around a circumference of the cylindrical body.

5. The catheter according to claim 1, wherein the pull wire receptacle comprises a slot beginning at a proximal end of the cylindrical body and having a length extending along a longitudinal axis of the cylindrical body.

6. The catheter according to claim 1, further comprising:
a ring electrode positioned around the coupler; and
a conductor attached to the ring electrode and routed through a via of the plurality of vias into the central cavity.

7. The catheter according to claim 1, further comprising a handle secured to a proximal end of the catheter shaft, the handle including an actuator, and wherein a proximal end of the pull wire is secured to the actuator such that movement of the actuator applies a tensile force to the pull wire and the tensile force is transmitted to the catheter shaft through the connection between the pull wire and the coupler.

8. A method of manufacturing a catheter, comprising:
providing a catheter shaft, a tip electrode, a pull wire, a sensor, and a coupler, wherein the coupler comprises:
a cylindrical body having an annular wall that defines a central cavity;
a plurality of vias extending through the annular wall into the central cavity;
a pull wire receptacle; and
a sensor receptacle;
joining the catheter shaft, the tip electrode, and the coupler;
connecting the pull wire to the coupler by securing a distal end of the pull wire in the pull wire receptacle; and
securing the sensor within the sensor receptacle.

9. The method according to claim 8, wherein the sensor comprises a localization sensor.

10. The method according to claim 9, wherein the localization sensor comprises a plurality of magnetic coils.

11. The method according to claim 8, further comprising:
placing a ring electrode around the coupler;
attaching a conductor to the ring electrode; and
routing the conductor through a via of the plurality of vias and into the central cavity.

12. The method according to claim 8, further comprising:
securing a handle to a proximal end of the catheter shaft, the handle including an actuator; and
connecting the pull wire to the handle by securing a proximal end of the pull wire to the actuator such that movement of the actuator applies a tensile force to the pull wire and the tensile force is transmitted to the catheter shaft through the connection between the pull wire and the coupler.

13. A coupler for securing a tip electrode, a sensor, and a pull wire to a catheter shaft, comprising:
a cylindrical body having an annular wall that defines a central cavity;
a plurality of vias extending through the annular wall into the central cavity;
a pull wire receptacle; and
a sensor receptacle.

14. The coupler according to claim 13, wherein the sensor receptacle comprises a slot that extends both along a longitudinal axis of the cylindrical body and around a circumference of the cylindrical body.

15. The coupler according to claim 13, wherein the pull wire receptacle comprises a slot that begins at a proximal end of the cylindrical body and has a length extending along a longitudinal axis of the cylindrical body.
